(19) United States
(12) Patent Application Publication
(10) Pub. No.: US 2004/0133100 A1
(43) Pub. Date: Jul. 8, 2004

(54) Novel risk assessment method based upon coronary calcification distribution pattern imaged by computed tomography

(76) Inventors: Morteza Naghavi, Houston, TX (US); S. Ward Casscells, Houston, TX (US); James T. Willerson, Houston, TX (US)

Correspondence Address: DUANE, MORRIS, LLP, SUITE 3150, 3200 SOUTHWEST FREEWAY, HOUSTON, TX, 77046, US

(21) Appl. No.: 10/645,970

(22) Filed: Aug. 22, 2003

Related U.S. Application Data
(60) Provisional application No. 60/405,322, filed on Aug. 23, 2002.

(51) Int. Cl. A61B 6/00

(52) U.S. Cl. 600/425; 128/920; 378/4; 382/128

(57) ABSTRACT

The present invention relates to a system and method for using an analysis of data generated during a scan of a patient to aid in assessment of coronary risk based upon coronary calcification. In an embodiment, a region of interest in a patient is scanned using computed tomography (CT). CT generated data resulting from the scanning are stored where the data comprise calcification data. The data are analyzed to determine a distribution of calcification in the patient and the patient's risk of cardiovascular disease based upon the analysis is assessed. It is emphasized that this abstract is provided to comply with the rules requiring an abstract which will allow a searcher or other reader to quickly ascertain the subject matter of the technical disclosure. It is submitted with the understanding that it will not be used to interpret or limit the scope or meaning of the claims.

### PRIORITY INFORMATION

This application claims the benefit of U.S. Provisional Application No. 60/405,322 filed on Aug. 23, 2002.

### FIELD OF INVENTION

The present invention relates generally to the field of coronary risk assessment. More particularly, the present invention relates to a system and method for using an analysis of data generated during a scan of a patient to aid in assessment of coronary risk based upon coronary calcification.

### BACKGROUND OF THE INVENTION

Coronary artery disease is the leading cause of death in the United States. While an office-based risk factor assessment is currently the reference standard for prediction of cardiac risk, invasive and noninvasive imaging techniques may be preferable to assess atherosclerotic vessels. Most of the standard techniques identify luminal diameter, stenosis, wall thickness, and plaque volume; however, none can characterize plaque composition and therefore identify the high-risk plaques.

Coronary calcium is clearly linked with coronary atherosclerosis. Electron beam computed tomography (EBCT) can be used to document the presence of and monitor the progression of atherosclerotic coronary artery calcifications in the general adult population. EBCT can accurately identify calcium in the coronary tree non-invasively. In population studies, populations with higher calcium scores have more calcium events. Interpretation of the clinical importance of different coronary artery calcium scores in the same subject is dependent on several factors, which include measurement variation and expected rate of progression of coronary artery calcium.

Coronary calcium scores do not correlate well with the degree of luminal narrowing. The calcified plaque is most likely not at the highest risk, rather the presence of calcium indicates the presence of atherosclerosis and, therefore, the likelihood that non-calcified “unstable” plaques may be present. The transition zone between calcified and non-calcified plaques may be at most risk of rupture due to the shear stresses occurring from blood moving through these transition zones.

The quantity of coronary artery calcium as detected with EBCT is indicative of plaque mass, and the likelihood of coronary obstruction and future coronary events is independent of other risk factors. Screening for coronary artery disease with EBCT offers a complimentary way of detecting early atherosclerosis in asymptomatic patients.

Coronary calcium is three to nine times higher in persons with fatal or nonfatal myocardial infarction than in age-matched controls, and four observational outcomes studies have demonstrated that the EBCT-derived coronary calcium score predicts fatal and nonfatal myocardial infarction. In symptomatic persons undergoing cardiac catheterization, EBCT is more closely associated with the severity of coronary atherosclerosis than are standard coronary risk factors. Preliminary evidence in asymptomatic persons indicates that the coronary calcium score also predicts coronary disease events more accurately than standard risk factors.

There is a need for a screening test that would allow early identification of coronary artery disease in its asymptomatic stage using calcium as a screening tool.

### SUMMARY OF THE INVENTION

A system for assessing coronary risk based upon coronary calcification may comprise a scanner adapted to detect a characteristic of a region of interest in a patient; a data store operatively coupled to the scanner and adapted to receive and store data generated by the scanner; and a data analyzer operatively coupled to the data store, wherein the data analyzer further comprises a scoring module adapted to determine distribution of the scanned characteristic of the region of interest in the patient.

Coronary risk based upon coronary calcification may be assessed by scanning a region of interest in a patient using computed tomography (CT); storing CT generated data resulting from said scanning, the data comprising calcification data; analyzing the data to determine a distribution of calcification in the patient; and assessing the patient's risk of cardiovascular disease based upon said analyzing.

In an alternative embodiment, coronary risk based upon coronary calcification may be assessed by scanning a region of interest in a patient using computed tomography (CT); storing CT generated data resulting from said scanning, the data comprising calcification data related to calcification of a blood vessel; generating scoring data representative of a statistical distribution of calcification in the blood vessel using the calcification data; and assessing the patient's risk of cardiovascular disease using the scoring data.

This summary is not to be interpreted as limiting the scope of these inventions which are limited only by the claims herein.

### BRIEF DESCRIPTION OF THE DRAWINGS

FIG. 1 is a schematic diagram of a preferred embodiment of a system for coronary risk assessment;

FIG. 2 is a flowchart of a first preferred embodiment of a method of coronary risk assessment; and

FIG. 3 is a flowchart of a second preferred embodiment of a method of coronary risk assessment.

### DETAILED DESCRIPTION OF PREFERRED EMBODIMENTS OF THE INVENTION

As used herein, that which is described as software may be equivalently implemented as hardware.

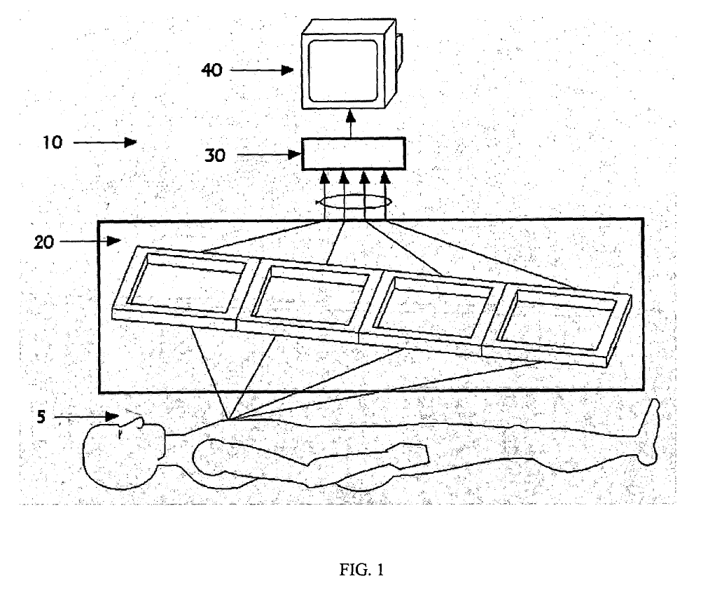

Referring now to FIG. 1, the preferred embodiment illustrated in system 10 may be used for assessing coronary risk based upon coronary calcification. In a preferred embodiment, system 10 comprises scanner 20; data store 30; and data analyzer 40. Data analyzer 40 may further comprise scoring module 42 software which is adapted to determine a distribution of the scanned characteristic of the region of interest in patient 5.

Scanner 20 is adapted to detect a desired characteristic of a region of interest in patient 5. In a preferred embodiment, the characteristic of the region of interest in the patient is calcification of a blood vessel, e.g. a coronary artery. Scanner 20 may comprise a computed tomography (CT) scanner, an electron beam computed tomography (EBCT) scanner, a multisection spiral CT, or the like, or a combination thereof. In certain currently contemplated embodiments, scanner 20 may further comprise multiple detectors.

Data store 30 is operatively coupled to scanner 20 and adapted to receive and store data generated by scanner 20. Data store 30 may comprise a persistent data store, e.g. a magnetic medium, an electronic medium, an optical medium, an electro-optic medium, or the like, or a combination thereof, and/or a transient data store, e.g. random access memory (RAM).

Data analyzer 40 may be any suitable computing device capable of hosting scoring module 42 (not illustrated in the figures) and interfacing with data store 30 to retrieve and, optionally, store data, e.g. a personal computer, a handheld computer, a personal digital assistant, or the like.

Scoring module 42 (not illustrated in the figures) or other software executing in data analyzer 40 may be further adapted to perform calculations on the data, e.g. perform statistical analyses such as determination of a mean, a median, a mode, a standard deviation, a range, a coefficient of variation, skew, kurtosis, or the like, or a combination thereof.

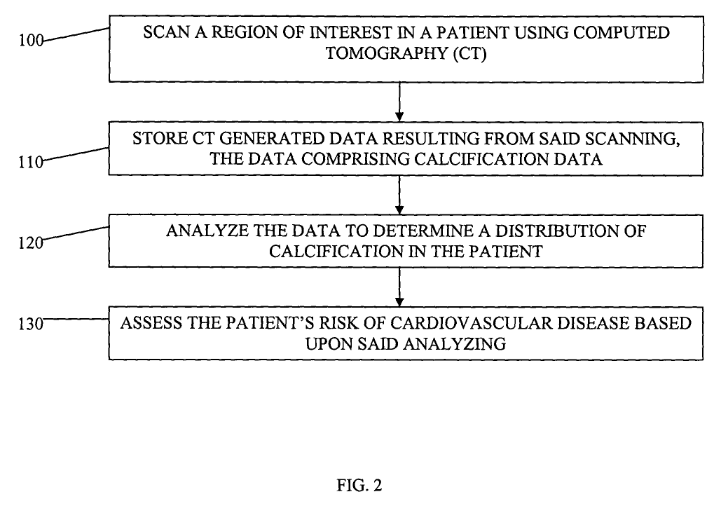

A preferred method embodiment of the present invention is illustrated in FIG. 2. In this embodiment, coronary risk may be assessed based upon coronary calcification by scanning a region of interest in patient 5, illustrated in FIG. 1, using computed tomography (CT), as illustrated in block 100 of FIG. 2. Scanning may use electron beam computed tomography (EBCT) and/or multiple detectors. Additionally, scanning may be performed on at least two slices of the body of patient 5. In certain contemplated embodiments, scanning may be done with multisection spiral CT.

The method of FIG. 2 further comprises storing CT generated data resulting from this scanning where the data comprise calcification data, as illustrated in block 110 of FIG. 2. Storing may comprise storing data for multiple pixels in the scanned region.

The CT generated data may then be analyzed, as illustrated in block 120 of FIG. 2, such as by using scoring module 42 of FIG. 1 to determine a distribution of calcification in patient 5. In a preferred embodiment, analyzing comprises determining proximal and distal artery calcification, determining the distribution of calcification in multiple coronary branches of the scanned region, determining concentric and eccentric calcification, determining changes in calcification density, determining the size of plaque in calcified areas, determining the shape of plaque in calcified areas, determining the density of plaque in multiple calcified areas, or the like, or a combination thereof.

Analyzing may further comprise calculating a statistical characteristic of the data, e.g. a mean, a median, a mode, a standard deviation, a range, a coefficient of variation, skew, kurtosis, or the like, or a combination thereof. The data and the statistical characteristic may be used to map a plurality of sections of a coronary artery as a function of calcification of each of the plurality of sections.

The method of FIG. 2 further comprises assessing the risk of cardiovascular disease for the patient based upon the analyzing, as illustrated in block 130 of FIG. 2. By way of example and not limitation, output from scoring module 42 may be presented on a display associated with data analyzer 40, e.g. a monitor or display or printer, for use by a trained medical professional. By way of further example and not limitation, an area of abrupt change in regional coronary elasticity may be categorized an as a high-risk region.

Assessing this risk of cardiovascular disease may further comprise using the map to determine progression of plaque and using the determined plaque progression to categorize the patient's risk of cardiovascular disease.

Analyzing may comprise calculating energy attenuation for each pixel in the scanned region, e.g. calculating an x-ray attenuation coefficient CT number for each pixel that is above a predetermined threshold. In an embodiment, the predetermined threshold is 130 Hounsfield units.

Determined changes in calcification density may be used when assessing the patient's risk of cardiovascular disease, e.g. by relating differing calcification densities in place to an outcome of a lesion.

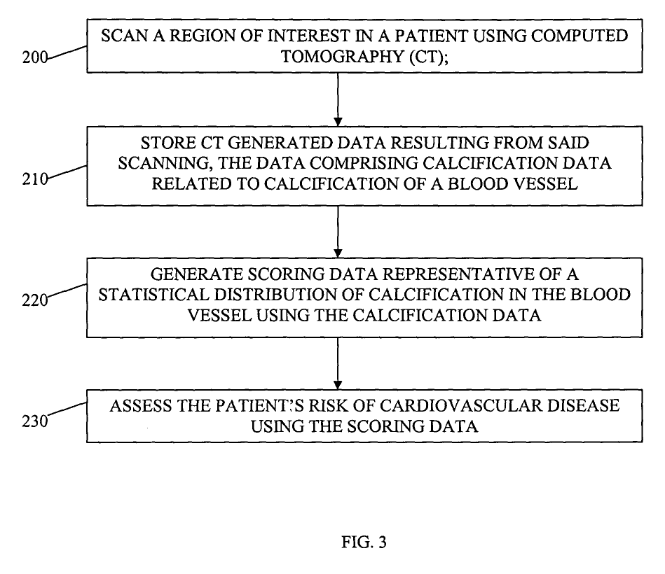

In a second preferred embodiment, as illustrated in FIG. 3, assessment of coronary risk may be based upon coronary calcification by scanning a region of interest in patient 5 using computed tomography (CT), as illustrated in block 200 of FIG. 3. Scanning may use electron beam computed tomography (EBCT) and/or multiple detectors. Further, scanning may be performed on at least two slices of the body of patient 5. In currently contemplated embodiments, scanning may be done with multisection spiral CT.

CT generated data resulting from the scanning may be stored, as illustrated in block 210 of FIG. 3, where the data comprising calcification data related to calcification of a blood vessel. Storing may comprise storing the CT generated data for multiple pixels in the scanned region.

Scoring data representative of a statistical distribution of calcification in the blood vessel using the calcification data may be generated, as illustrated in block 220 of FIG. 3. Generating scoring data may comprise determining proximal and distal artery calcification, determining the distribution of calcification in multiple coronary branches of the scanned region, determining concentric and eccentric calcification, determining changes in calcification density, determining the size of plaque in calcified areas, determining the shape of plaque in calcified areas, determining the density of plaque in multiple calcified areas, or the like, or a combination thereof.

The generation of the scoring data may further comprise calculating energy attenuation for each pixel in the scanned region, e.g. calculating an x-ray attenuation coefficient CT number for each pixel that is above a predetermined threshold. In an embodiment, the predetermined threshold is 130 Hounsfield units.

The statistical distribution may further comprise a mean, a median, a mode, a standard deviation, a range, a coefficient of variation, skew, or kurtosis, or the like, or a combination thereof.

The patient's risk of cardiovascular disease may be assessed using the scoring data, as illustrated in block 230 of FIG. 3. If changes in calcification density are determined, the determined changes in calcification density may be used when assessing the risk of cardiovascular disease for patient 5, e.g. by relating differing calcification densities in place to an outcome of a lesion. For example, an area of abrupt change in regional coronary elasticity may be categorized as a high-risk region.

In another preferred embodiment, assessments may be aided by using the CT generated data and the scoring data to map a plurality of sections of the blood vessel as a function of statistical distribution of calcification of each of the plurality of sections. The map may be used to determine progression of plaque and the determined plaque progression used to categorize the risk of cardiovascular disease for patient 5.

It will be understood that various changes in the details, materials, and arrangements of the parts which have been described and illustrated above in order to explain the nature of this invention may be made by those skilled in the art without departing from the principle and scope of the invention as recited in the appended claims.

### STATEMENT OF INDUSTRIAL USE

The present invention may be used for coronary risk assessment using an analysis of data generated during a scan of a patient to aid in assessment of coronary risk based upon coronary calcification.

What is claimed is:

1. A method of assessing coronary risk based upon coronary calcification, comprising:
a. scanning a region of interest in a patient using computed tomography (CT);
b. storing CT generated data resulting from said scanning, the data comprising calcification data;
C. analyzing the data to determine a distribution of calcification in the patient; and
d. assessing the patient's risk of cardiovascular disease based upon said analyzing.

2. The method of claim 1, wherein said scanning uses electron beam computed tomography (EBCT).

3. The method of claim 1, wherein said scanning uses multiple detectors.

4. The method of claim 1, wherein said scanning is performed on at least two slices of the patient's body.

5. The method of claim 1, wherein said scanning is done with multisection spiral CT.

6. The method of claim 1, wherein said storing comprises storing data for multiple pixels in the scanned region.

7. The method of claim 6, wherein said analyzing comprises calculating energy attenuation for each pixel in the scanned region.

8. The method of claim 7, wherein said calculating comprises calculating an x-ray attenuation coefficient CT number for each pixel that is above a predetermined threshold.

9. The method of claim 8, wherein said predetermined threshold is 130 Hounsfield units.

10. The method of claim 1, wherein said analyzing comprises at least one of (i) determining proximal and distal artery calcification, (ii) determining the distribution of calcification in multiple coronary branches of the scanned region, (iii) determining concentric and eccentric calcification, (iv) determining changes in calcification density, (v) determining the size of plaque in calcified areas, (vi) determining the shape of plaque in calcified areas, or (vii) determining the density of plaque in multiple calcified areas.

11. The method of claim 10, further comprising using the determined changes in calcification density when assessing the patient's risk of cardiovascular disease by relating differing calcification densities in place to an outcome of a lesion.

12. The method of claim 1, wherein said analyzing further comprises calculating a statistical characteristic of the data.

13. The method of claim 12, wherein the calculating a statistical characteristic further comprises calculating at least one of (i) mean, (ii) median, (iii) mode, (iv) standard deviation, (v) range, (vi) coefficient of variation, (vii) skew, or (viii) kurtosis.

14. The method of claim 12, further comprising using the data and the statistical characteristic to map a plurality of sections of a coronary artery as a function of calcification of each of the plurality of sections.

15. The method of claim 14, wherein assessing the patient's risk of cardiovascular disease based upon said analyzing further comprises:
a. using the map to determine progression of plaque; and
b. using the determined plaque progression to categorize the patient's risk of cardiovascular disease.

16. The method of claim 15, further comprising categorizing an area of abrupt change in regional coronary elasticity as a high-risk region.

17. A method of assessing coronary risk based upon coronary calcification, comprising:
a. scanning a region of interest in a patient using computed tomography (CT);
b. storing CT generated data resulting from said scanning, the data comprising calcification data related to calcification of a blood vessel;
C. generating scoring data representative of a statistical distribution of calcification in the blood vessel using the calcification data; and
d. assessing the patient's risk of cardiovascular disease using the scoring data.

18. The method of claim 17, wherein said scanning uses at least one of (i) electron beam computed tomography (EBCT) or (ii) multiple detectors.

19. The method of claim 17, wherein said scanning is performed on at least two slices of the patient's body.

20. The method of claim 17, wherein said scanning is done with multisection spiral CT.

21. The method of claim 17, wherein said storing comprises storing the CT generated data for multiple pixels in the scanned region.

22. The method of claim 21, wherein said generating scoring data further comprises calculating energy attenuation for each pixel in the scanned region.

23. The method of claim 22, wherein said calculating further comprises calculating an x-ray attenuation coefficient CT number for each pixel that is above a predetermined threshold.

24. The method of claim 23, wherein said predetermined threshold is 130 Hounsfield units.

25. The method of claim 17, wherein said generating scoring data further comprises at least one of (i) determining proximal and distal artery calcification, (ii) determining the distribution of calcification in multiple coronary branches of the scanned region, (iii) determining concentric and eccentric calcification, (iv) determining changes in calcification density, (v) determining the size of plaque in calcified areas, (vi) determining the shape of plaque in calcified areas, or (vii) determining the density of plaque in multiple calcified areas.

26. The method of claim 25, further comprising using the determined changes in calcification density when assessing the patient's risk of cardiovascular disease by relating differing calcification densities in place to an outcome of a lesion.

27. The method of claim 17, wherein said statistical distribution further comprises at least one of (i) a mean, (ii) a median, (iii) a mode, (iv) a standard deviation, (v) a range, (vi) a coefficient of variation, (vii) skew, or (viii) kurtosis.

28. The method of claim 27, further comprising using the CT generated data and the scoring data to map a plurality of sections of the blood vessel as a function of statistical distribution of calcification of each of the plurality of sections.

29. The method of claim 28, wherein assessing the patient's risk of cardiovascular disease based upon said analyzing further comprises:
a. using the map to determine progression of plaque; and
b. using the determined plaque progression to categorize the patient's risk of cardiovascular disease.

30. The method of claim 31, further comprising categorizing an area of abrupt change in regional coronary elasticity as a high-risk region.

31. A system for assessing coronary risk based upon coronary calcification, comprising:
a. a scanner adapted to detect a characteristic of a region of interest in a patient;
b. a data store operatively coupled to the scanner and adapted to receive and store data generated by the scanner; and
c. a data analyzer operatively coupled to the data store, wherein the data analyzer further comprises a scoring module adapted to determine distribution of the scanned characteristic of the region of interest in the patient.

32. The system of claim 33, wherein the scanner comprises at least one of (i) a computed tomography (CT) scanner, (ii) an electron beam computed tomography (EBCT) scanner, or (iii) a multisection spiral CT.

33. The system of claim 33, wherein the scanner comprises multiple detectors.

34. The system of claim 33, wherein the characteristic of the region of interest in the patient is calcification of a blood vessel.
